(19) United States
(12) Patent Application Publication
(10) Pub. No.: US 2023/0357113 A1
(43) Pub. Date: Nov. 9, 2023

(54) TAPINAROF POLYMORPHS

(71) Applicant: Sol-Gel Technologies Ltd., Ness Ziona (IL)

(72) Inventors: Moshe Arkin, Kfar Shmaryahu (IL); Natalie Utsis, Beer Sheva (IL); Ariel Mitelman, Beer Sheva (IL); Bar Havazelt, Beer Sheva (IL); Shai Dan, Beer Sheva (IL)

(73) Assignee: Sol-Gel Technologies Ltd., Ness Ziona (IL)

(21) Appl. No.: 18/144,297

(22) Filed: May 8, 2023

Related U.S. Application Data
(60) Provisional application No. 63/339,470, filed on May 8, 2022.

(51) Int. Cl. C07C 37/84

(52) U.S. Cl. CPC C07C 37/84; C07B 2200/13

(57) ABSTRACT

The present invention relates to crystalline polymorphs of Tapinarof, e.g., Tapinarof crystalline Form B, Form C, and Form D, pharmaceutical compositions comprising the same, processes for preparation thereof, and uses thereof for treatment of skin disorders.

### CROSS REFERENCE TO RELATED APPLICATIONS

This application claims the benefit of U.S. Ser. No. 63/339,470, filed on May 8, 2022 (and entitled TAPINAROF POLYMORPHS), which is incorporated in its entirety herein by reference.

### FIELD OF THE INVENTION

The present invention relates to crystalline polymorphs of Tapinarof, pharmaceutical compositions comprising the same, processes for preparation thereof, and uses thereof for treatment of skin disorders.

### BACKGROUND OF THE INVENTION

Tapinarof, has a chemical name of 3,5-dihydroxy-4-isopropyl-trans-stilbene and the chemical structure:

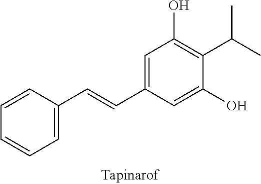 [SMILES: CC(C)c1c(O)cc(/C=C/c2ccccc2)cc1O]

Polymorphism, the occurrence of different crystalline forms, is a property of some molecules and molecular complexes. A single molecule, like Tapinarof, may give rise to a variety of polymorphs having distinct crystal structures and physical properties, e.g., melting point, thermal behaviours (measured by thermogravimetric analysis—“TGA” or differential scanning calorimetry—“DSC”), X-ray diffraction (XRD) pattern, infrared absorption fingerprint, and solid state (¹³C) NMR spectrum. One or more of these techniques may be used to distinguish different polymorphic forms of a chemical compound.

The skin is a barrier that controls the body's moisture and prevents the penetration of microorganisms because the skin is composed of superficial layers of epidermis and stratum corneum that provide most of the skin barrier properties. The stratum corneum consists of layers of overlapping cell plates containing keratin.

Usually, drug absorption via the skin is transcellular, that is, a passive diffusion process, which depends on the efficacy of the epidermal barrier and the nature of the drug itself. Drugs having low molecular weight of less than about 800 Daltons with high water and lipid solubility demonstrate the greatest skin penetration. Different crystalline forms or polymorphs of the same pharmaceutical compounds have different aqueous solubility hence different absorption profile.

Discovering new solid-state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid-state forms of a pharmaceutically useful compound can also provide an opportunity to improve the performance characteristics of a pharmaceutical product. For at least these reasons, there is a need for additional solid-state forms of Tapinarof. Moreover, the processes for preparing such forms should be suitable for industrial production and not cumbersome. The present invention provides such novel crystalline forms and processes for their preparations.

### SUMMARY OF THE INVENTION

In one aspect, the present invention relates to a crystalline Form B of Tapinarof, characterized by an X-ray powder diffraction pattern exhibiting characteristic diffraction (XRPD) peaks at 7.7, 11.0, and 22.6 (±0.2) ° 2θ.

In another aspect, the present invention relates to a crystalline Form C of Tapinarof, characterized by an X-ray powder diffraction pattern exhibiting characteristic diffraction (XRPD) peaks at 5.2, 10.5, 12.6, 15.8, and 22.9 (±0.2) ° 2θ.

In another aspect, the present invention relates to a crystalline Form D of Tapinarof, characterized by an X-ray powder diffraction pattern exhibiting characteristic diffraction (XRPD) peaks at 8.8, 10.8, 13.2, 19.8, and 20.1 (±0.2) ° 2θ. In some embodiments, the crystalline Form D of Tapinarof of the invention is further characterized by one or more peaks at 4.4, 16.5, 18.6, 21.7, and 26.5 (±0.2) ° 2θ.

In a further aspect, the present invention provides pharmaceutical compositions comprising a crystalline form of Tapinarof of the present invention, e.g., a crystalline Form B of Tapinarof, a crystalline Form C of Tapinarof, or a crystalline Form D of Tapinarof, and at least one pharmaceutically acceptable excipient.

In yet another aspect, the present invention provides a Tapinarof compound, wherein the compound has a cis isomer content of 0.1% or less, wherein the compound is at least 85% pure by weight, wherein the compound is at least 85% Form B by weight, characterized by an x-ray powder diffraction pattern having peaks at 7.7, 11.0, and 22.6 (±0.2) ° 2θ.

The present invention further provides a Tapinarof compound, wherein the compound has a cis isomer content of 0.1% or less, wherein the compound is at least 85% pure by weight, wherein the compound is at least 85% Form C, characterized by an x-ray powder diffraction pattern having peaks at 5.2, 10.5, 12.6, 15.8, and 22.9 (±0.2) ° 2θ.

In a further aspect, the present invention provides a Tapinarof compound, wherein the compound has a cis isomer content of 0.1% or less, wherein the compound is at least 85% pure by weight, wherein the compound is at least 85% Form D, characterized by an x-ray powder diffraction pattern having peaks at 8.8, 10.8, 13.2, 19.8, and 20.1 (±0.2) ° 2θ.

In another aspect, the present invention provides methods of using a crystalline form of Tapinarof of the present invention, e.g., a crystalline Form B of Tapinarof, a crystalline Form C of Tapinarof, or a crystalline Form D of Tapinarof, or a pharmaceutical composition comprising thereof, or a Tapinarof compound of the invention in the treatment of a disease or condition for which Tapinarof provides therapeutic benefits to the subject in need thereof, such as, treatment of psoriasis (selected from plaque psoriasis, flexural/inverse psoriasis, scalp psoriasis, sebopsoriasis, nail psoriasis and genital psoriasis); dermatitis (also known as eczema) selected from atopic dermatitis, contact dermatitis, dyshidrotic dermatitis, nummular dermatitis and seborrheic dermatitis; acne selected from acne vulgaris, papulopustular acne and nodular acne; rosacea selected from erythematotelangiectatic rosacea, papulopustular rosacea, rhinophyma rosacea and ocular rosacea; ichthyosis selected from ichthyosis vulgaris, hereditary ichthyosis, acquired ichthyosis; scaling skin; imbalance of skin barrier; hidradenitis suppurativa; tinea selected from tinea pedis, tinea capitis, tinea barbae, tinea faciei, tinea corporis, tinea manum, tinea cruris and tinea interdigital; hereditary palmoplantar keratoderma; actinic keratosis; a keratinization skin disorder; a keratinization mucosal disorder; Gorlin syndrome; Palmoplantar Keratoderma (PPK); and Olmsted syndrome by topical or intralesional administration.

### BRIEF DESCRIPTION OF THE DRAWINGS

FIG. 1 depicts the XRPD pattern of crystalline Form B of Tapinarof.

FIG. 2 depicts the XRPD pattern of crystalline Form C of Tapinarof.

FIG. 3 depicts the XRPD pattern of crystalline Form D of Tapinarof.

### DETAILED DESCRIPTION OF THE INVENTION

In one aspect, the present invention relates to a crystalline Form B of Tapinarof, characterized by an X-ray powder diffraction pattern exhibiting characteristic diffraction (XRPD) peaks at 7.7, 11.0, and 22.6 (±0.2) ° 2θ.

In some embodiments, the crystalline Form B is further characterized by one or more peaks at 6.1, 9.9, 15.5, 20.0, and 21.9 (±0.2) ° 2θ.

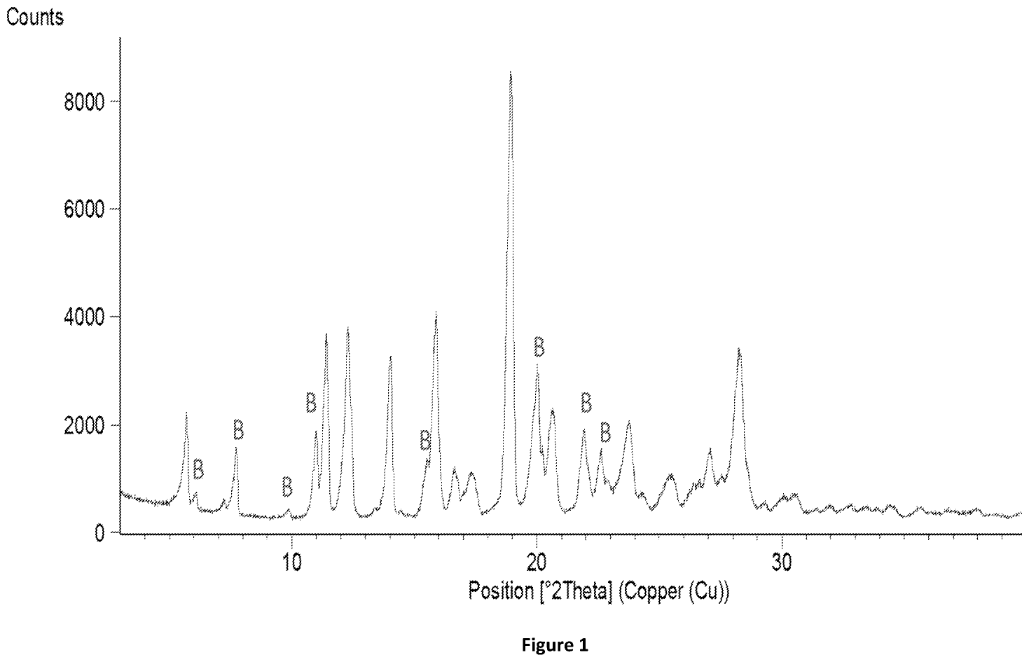

In some embodiments, the crystalline Form B has the X-ray powder diffraction peak positions and intensities as depicted in FIG. 1.

In some embodiments, the crystalline Form B is an anhydrous form. In some embodiments, the crystalline Form B is a non-solvated form.

In some embodiments, the present invention provides a pharmaceutical composition comprising a therapeutically effective amount of the crystalline Form B of Tapinarof and at least one pharmaceutically acceptable excipient. In some embodiments, the pharmaceutical composition comprises the crystalline Form B of Tapinarof of the invention in an amount of from about 0.1% w/w to about 10.0% w/w. In other embodiments, the pharmaceutical composition comprises the crystalline Form B of Tapinarof in an amount of 0.25%, 0.5%, 1%, or 5% w/w.

In one aspect, the present invention provides a Tapinarof compound, wherein the compound is about 85% pure by weight and wherein the compound is at least 85% Form B by weight. In some embodiments, the compound is at least 86% pure by weight. In some embodiments, the compound is at least 87% pure by weight. In some embodiments, the compound is at least 88% pure by weight. In some embodiments, the compound is at least 89% pure by weight. In some embodiments, the compound is at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 85% Form B by weight, or at least 86% Form B by weight, or at least 87% Form B by weight, or at least 88% Form B by weight, or at least 89% Form B by weight, or at least 90% Form B by weight, or at least 91% Form B by weight, or at least 92% Form B by weight, or at least 93% Form B by weight, or at least 94% Form B by weight, or at least 95% Form B by weight, or at least 96% Form B by weight, or at least 97% Form B by weight, or at least 98% Form B by weight, or at least 99% Form B by weight. In some embodiments, the compound is about 96% Form B by weight, about 97% Form B by weight, about 98% Form B by weight, or about 99% Form B by weight. In some embodiments, the compound is at least 99% Form B by weight.

In one aspect, the present invention provides a Tapinarof compound, wherein the compound is about 98% pure by weight and wherein the compound is at least 95% Form B of the invention. In some embodiments, the Tapinarof compound is about 96% Form B, about 97% Form B, about 98% Form B, or about 99% Form B. In some embodiments, the Tapinarof compound is at least 99% Form B. In some embodiments, the present invention provides a pharmaceutical composition comprising a therapeutically effective amount of the Tapinarof compound of the invention having at least 95% Form B and at least one pharmaceutically acceptable excipient. In some embodiment, the compound Tapinarof in the pharmaceutical composition has a chemical purity of at least 85% by weight. In other embodiments, the Tapinarof compound in the pharmaceutical composition has a chemical purity of at least 85%, 86%, 87%, 88%, 89%, 90%, 91%, 92%, 93%, 94%, 95%, 96%, 97%, 98%, or 99% by weight. In other embodiments, the pharmaceutical composition comprising the Tapinarof compound having at least 85%, 86%, 87%, 88%, 89%, 90%, 91%, 92%, 93%, 94%, 95%, 96%, 97%, 98%, or 99% crystalline Form B by weight and a pharmaceutical composition comprising thereof.

In one aspect, the present invention provides Tapinarof compound, wherein the compound has a cis isomer content of 0.1% or less, wherein the compound is at least 85% pure by weight, wherein the compound is at least 85% Form B by weight, characterized by an x-ray powder diffraction pattern having peaks at 7.7, 11.0, and 22.6 (±0.2) ° 2θ. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 85% Form B by weight, or at least 86% Form B, or at least 87% Form B, or at least 88% Form B, or at least 89% Form B, or at least 90% Form B, or at least 91% Form B by weight, or at least 92% Form B by weight, or at least 93% Form B by weight, or at least 94% Form B by weight, or at least 95% Form B by weight, or at least 96% Form B by weight, or at least 97% Form B by weight, or at least 98% Form B by weight, or at least 99% Form B by weight.

In some embodiments, the present invention provides Tapinarof compound, wherein the compound has a cis isomer content of 0.1% or less, wherein the compound is at least 98% pure by weight, wherein the compound is at least 95% Form B, characterized by an x-ray powder diffraction pattern having peaks at 7.7, 11.0, and 22.6 (±0.2) ° 2θ.

In another aspect, the present invention relates to a crystalline Form C of Tapinarof, characterized by an X-ray powder diffraction pattern exhibiting characteristic diffraction (XRPD) peaks at 5.2, 10.5, 12.6, 15.8, and 22.9 (±0.2) ° 2θ.

In some embodiments, the crystalline Form C of Tapinarof of the invention is further characterized by one or more peaks at 11.4, 25.5, 26.5, 27.1, 31.6 (±0.2) ° 2θ.

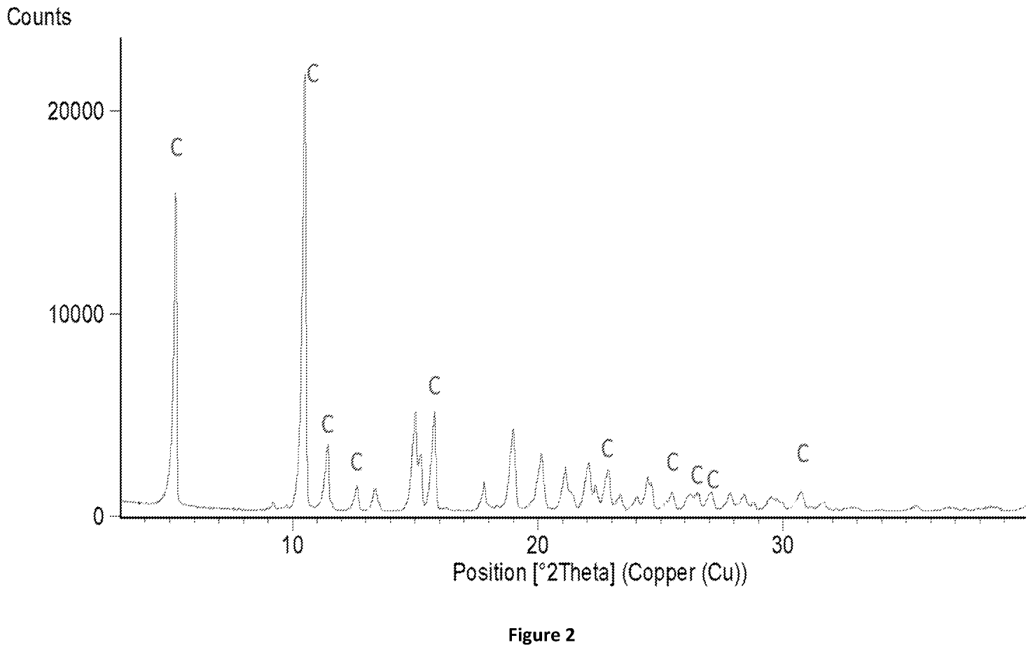

In some embodiments, the crystalline Form C of Tapinarof of the invention has the X-ray powder diffraction peak positions and intensities as depicted in FIG. 2.

In some embodiments, the crystalline Form C of Tapinarof of the invention is an isopropanol solvate form. In some embodiments, the crystalline Form C of Tapinarof of the invention is an anhydrous form.

In some embodiments, the present invention provides a pharmaceutical composition comprising a therapeutically effective amount of the crystalline Form C of Tapinarof of the invention and at least one pharmaceutically acceptable excipient. In some embodiments, the pharmaceutical composition comprises the crystalline Form C of Tapinarof in an amount of from about 0.25% w/w to about 10.0% w/w. In other embodiments, the pharmaceutical composition comprises the crystalline Form C of Tapinarof in an amount of 0.25%, 0.5%, 1%, or 5% w/w.

In a further aspect, the present invention provides a Tapinarof compound, wherein the compound is about 85% pure by weight and wherein the compound is at least 85% Form C by weight. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 85% Form C by weight, or at least 86% Form C by weight, or at least 87% Form C by weight, or at least 88% Form C by weight, or at least 89% Form C by weight, or at least 90% Form C by weight, or at least 91% Form C by weight, or at least 92% Form C by weight, or at least 93% Form C by weight, or at least 94% Form C by weight, or at least 95% Form C by weight, or at least 96% Form C by weight, or at least 97% Form C by weight, or at least 98% Form C by weight, or at least 99% Form C by weight. In some embodiments, the compound is about 96% Form C by weight, about 97% Form C by weight, about 98% Form C by weight, or about 99% Form C by weight. In some embodiments, the compound is at least 99% Form C by weight.

In one aspect, the present invention provides a Tapinarof compound, wherein the compound is about 98% pure by weight and wherein the compound is at least 95% Form C of the invention. In some embodiments, the Tapinarof compound is about 96% Form C, about 97% Form C, about 98% Form C, or about 99% Form C. In some embodiments, the Tapinarof compound is at least 99% Form C. In some embodiments, the present invention provides a pharmaceutical composition comprising a therapeutically effective amount of the Tapinarof compound of the invention having at least 95% Form C and at least one pharmaceutically acceptable excipient. In some embodiment, the Tapinarof compound in the pharmaceutical composition has a chemical purity of at least 85%. In other embodiments, the Tapinarof compound in the pharmaceutical composition has a chemical purity of at least 85%, 86%, 87%, 88%, 89%, 90%, 91%, 92%, 93%, 94%, 95%, 96%, 97%, 98%, or 99%. In other embodiments, the pharmaceutical composition comprising the Tapinarof compound having at least 85%, 86%, 87, 88%, 89%, 90%, 91%, 92%, 93%, 94%, 95%, 96%, 97%, 98%, or 99% crystalline Form C and a pharmaceutical composition comprising thereof.

In another aspect, the present invention provides a Tapinarof compound, wherein the compound has a cis isomer content of 0.1% or less, wherein the compound is at least 85% pure by weight, wherein the compound is at least 85% Form C, characterized by an x-ray powder diffraction pattern having peaks at 5.2, 10.5, 12.6, 15.8, and 22.9 (±0.2) ° 2θ. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 85%% Form C by weight, or at least 86% Form C, or at least 87% Form C, or at least 88% Form C, or at least 89% Form C, or at least 90% Form C, or at least 91% Form C by weight, or at least 92% Form C by weight, or at least 93% Form C by weight, or at least 94% Form C by weight, or at least 95% Form C by weight, or at least 96% Form C by weight, or at least 97% Form C by weight, or at least 98% Form C by weight, or at least 99% Form C by weight.

In some embodiments, the present invention provides a Tapinarof compound, wherein the compound has a cis isomer content of 0.1% or less, wherein the compound is at least 98% pure by weight, wherein the compound is at least 95% Form C, characterized by an x-ray powder diffraction pattern having peaks at 5.2, 10.5, 12.6, 15.8, and 22.9 (±0.2) ° 2θ.

In another aspect, the present invention relates to a crystalline Form D of Tapinarof, characterized by an X-ray powder diffraction pattern exhibiting characteristic diffraction (XRPD) peaks at 8.8, 10.8, 13.2, 19.8, and 20.1 (±0.2) ° 2θ. In some embodiments, the crystalline Form D of Tapinarof of the invention is further characterized by one or more peaks at 4.4, 16.5, 18.6, 21.7, and 26.5 (±0.2) ° 2θ.

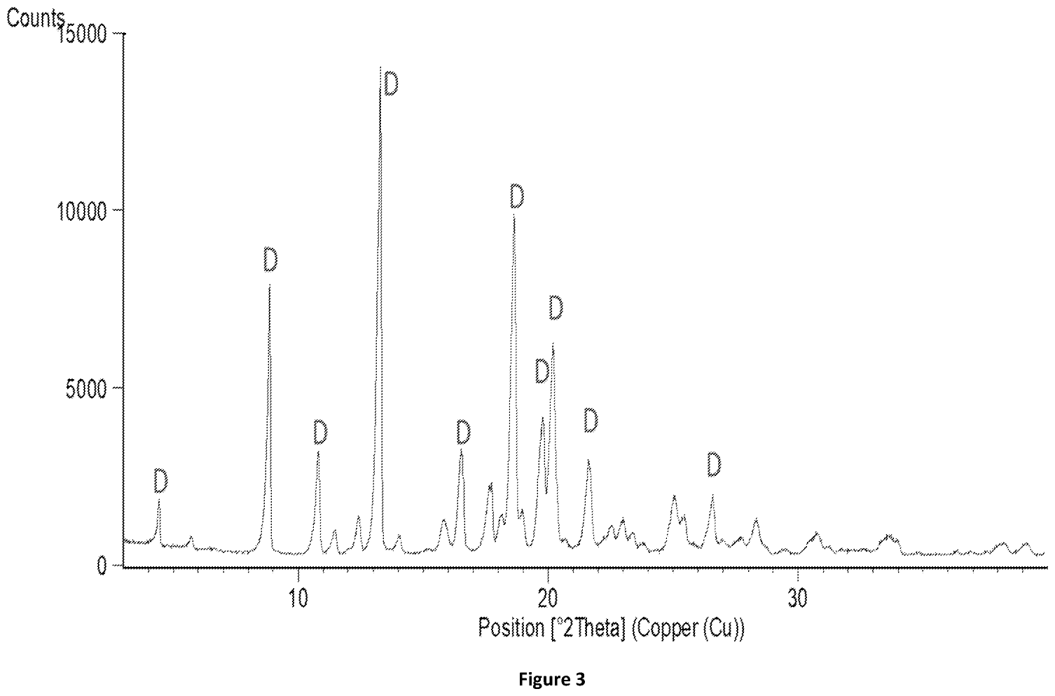

In some embodiments, the crystalline Form D of Tapinarof of the invention has the X-ray powder diffraction peak positions and intensities as depicted in FIG. 3.

In some embodiments, the crystalline Form D of Tapinarof of the invention is a methyl t-butyl ether solvate form. In other embodiments, the crystalline Form D of Tapinarof of the invention is an anhydrous form.

In some embodiments, the present invention provides a pharmaceutical composition comprising a therapeutically effective amount of the crystalline Form D of Tapinarof of the invention and at least one pharmaceutically acceptable excipient. In some embodiments, the pharmaceutical composition comprises the crystalline Form D of Tapinarof in an amount of from about 0.25% w/w to about 10.0% w/w. In other embodiments, the pharmaceutical composition comprises the crystalline Form D of Tapinarof in an amount of 0.25%, 0.5%, 1%, or 5% w/w.

In a further aspect, the present invention provides a Tapinarof compound, wherein the compound is at least 85% pure by weight and wherein the compound is at least 85% Form D by weight. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 85% Form D by weight, or at least 86% Form D by weight, or at least 87% Form D by weight, or at least 88% Form D by weight, or at least 89% Form D by weight, or at least 90% Form D by weight, or at least 91% Form D by weight, or at least 92% Form D by weight, or at least 93% Form D by weight, or at least 94% Form D by weight, or at least 95% Form D by weight, or at least 96% Form D by weight, or at least 97% Form D by weight, or at least 98% Form D by weight, or at least 99% Form D by weight. In some embodiments, the compound is about 96% Form D by weight, about 97% Form D by weight, about 98% Form D by weight, or about 99% Form D by weight. In some embodiments, the compound is at least 99% Form D by weight.

In one aspect, the present invention provides a Tapinarof compound, wherein the compound is about 98% pure by weight and wherein the compound is at least 95% Form D of the invention. In some embodiments, the Tapinarof compound is about 96% Form D, about 97% Form D, about 98% Form D, or about 99% Form D. In some embodiments, the Tapinarof compound is at least 99% Form D. In some embodiments, the present invention provides a pharmaceutical composition comprising a therapeutically effective amount of the Tapinarof compound of the invention having at least 95% Form D and at least one pharmaceutically acceptable excipient. In some embodiment, the Tapinarof compound in the pharmaceutical composition has a chemical purity of at least 85%. In other embodiments, the Tapinarof compound in the pharmaceutical composition has a chemical purity of at least 91%, 92%, 93%, 94%, 95%, 96%, 97%, 98%, or 99%. In other embodiments, the pharmaceutical composition comprising the Tapinarof compound having at least 85%, 86%, 87%, 88%, 89%, 90%, 91%, 92%, 93%, 94%, 95%, 96%, 97%, 98%, or 99% crystalline Form D and a pharmaceutical composition comprising thereof.

In another aspect, the present invention provides a Tapinarof compound, wherein the compound has a cis isomer content of 0.1% or less, wherein the compound is at least 85% pure by weight, wherein the compound is at least 85% Form D, characterized by an x-ray powder diffraction pattern having peaks at 8.8, 10.8, 13.2, 19.8, and 20.1 (±0.2) ° 2θ. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 85% Form D by weight, or at least 86% Form D by weight, or at least 87% Form D by weight, or at least 88% Form D by weight, or at least 89% Form D by weight, or at least 90% Form D by weight, or at least 91% Form D by weight, or at least 92% Form D by weight, or at least 93% Form D by weight, or at least 94% Form D by weight, or at least 95% Form D by weight, or at least 96% Form D by weight, or at least 97% Form D by weight, or at least 98% Form D by weight, or at least 99% Form D by weight.

In some embodiments, the present invention provides a Tapinarof compound, wherein the compound has a cis isomer content of 0.1% or less, wherein the compound is at least 98% pure by weight, wherein the compound is at least 95% Form D, characterized by an x-ray powder diffraction pattern having peaks at 8.8, 10.8, 13.2, 19.8, and 20.1 (±0.2) ° 2θ.

In one embodiment, this invention provides a process for the preparation of a crystalline Form B of Tapinarof of the invention. The starting materials in the process are produced by any suitable method, including synthesis methods known in the art.

In some embodiments, the process of the present invention produces a high purity of the crystalline Form B of Tapinarof. In some embodiments, the chemical purity of the crystalline polymorph (Form B) of Tapinarof of the invention is from about 85% to about 100% by weight. In some embodiments, the chemical purity of the crystalline polymorph (Form B) of Tapinarof is from about 90% to about 100% by weight. In some embodiments, the chemical purity of the crystalline polymorph (Form B) of Tapinarof is from about 95% to about 100% by weight. In another embodiments, the chemical purity of the crystalline polymorph (Form B) of Tapinarof is from about 96% to about 100% by weight. In another embodiment, the chemical purity of the crystalline polymorph (Form B) of Tapinarof is from about 97% to about 100% by weight. In another embodiment, the chemical purity of the crystalline polymorph (Form B) of Tapinarof is from about 98% to about 100% by weight. In another embodiment, the chemical purity of the crystalline polymorph (Form B) of Tapinarof is from about 99% to about 100% by weight. In another embodiment, the crystalline polymorph (Form B) of Tapinarof has a chemical purity of about 99% by weight. In some embodiments, the crystalline polymorph (Form B) of Tapinarof has a chemical purity of at least 99% by weight.

In one embodiment, this invention provides a crystalline polymorph (Form B) of Tapinarof, which is prepared by the process of the invention as described herein.

According to an embodiment of the present invention, there is provided a process for preparing Tapinarof crystalline Form B said process comprising:
(i) mixing crude Tapinarof and an organic solvent to provide a mixture,
(ii) heating and stirring the mixture of step (i) to obtain a solution,
(iii) cooling and stirring the solution to form a slurry, and
(iv) isolating the slurry and drying to obtain the crystalline Form B.

In some embodiments, the organic solvent for the process of the invention for Form B is a mixture of saturated hydrocarbon and an alkyl alcohol. In some embodiments, the saturated hydrocarbon is cyclohexane or methyl cyclohexane. In some embodiments, the alkyl alcohol is methanol, ethanol, n-propyl alcohol, isopropyl alcohol, n-butanol, t-butanol, pentanol, or hexanol.

In some embodiments, the organic solvent for the process of the invention is a mixture of methyl cyclohexane and isopropyl alcohol.

In some embodiments, the heating of step (ii) in the process of the invention is heating to a temperature of about 75-85° C. In other embodiments, the heating of step (ii) is heating to about 80° C.

In some embodiments, the cooling and stirring of step (iii) is first cooling and stirring at 25-30° C., followed by further cooling and stirring at 0° C. In some embodiments, the first cooling and stirring at 25-30° C. is for about 1-5 hours. In some embodiments, the further cooling and stirring at 0° C. is for about 1-3 hours.

In some embodiments, the slurry in the process of the invention for Form B is isolated by filtration.

In one embodiment, this invention provides a process for the preparation of a crystalline Form C of Tapinarof. The starting materials in the process are produced by any suitable method, including synthesis methods known in the art.

In some embodiments, the process of the present invention produces a high purity of the crystalline Form C of Tapinarof. In some embodiments, the chemical purity of the crystalline polymorph (Form C) of Tapinarof is from about 85% to about 100% by weight. In some embodiments, the chemical purity of the crystalline polymorph (Form C) of Tapinarof is from about 90% to about 100% by weight. In some embodiments, the chemical purity of the crystalline polymorph (Form C) of Tapinarof is from about 95% to about 100% by weight. In another embodiment, the chemical purity of the crystalline polymorph (Form C) of Tapinarof is from about 96% to about 100% by weight. In another embodiment, the chemical purity of the crystalline polymorph (Form C) of Tapinarof is from about 97% to about 100% by weight. In another embodiment, the chemical purity of the crystalline polymorph (Form C) of Tapinarof is from about 98% to about 100% by weight. In another embodiment, the chemical purity of the crystalline polymorph (Form C) of Tapinarof is from about 99% to about 100% by weight. In another embodiment, the crystalline polymorph (Form C) of Tapinarof has a chemical purity of about 99% by weight. In some embodiments, the crystalline polymorph (Form C) of Tapinarof of the invention has a chemical purity of at least 99% by weight.

In one embodiment, this invention provides a crystalline polymorph (Form C) of Tapinarof, which is prepared by the process of the invention as described herein.

According to an embodiment of the present invention, there is provided a process for preparing Tapinarof crystalline Form C, said process comprising:
(i) mixing crude Tapinarof and an organic solvent to provide a mixture,
(ii) heating and stirring the mixture of step (i) to obtain a solution,
(iii) cooling and stirring the solution to form a slurry, and
(iv) isolating the slurry and drying to obtain the crystalline Form C.

In some embodiments, the organic solvent for the process of the invention for Form C is a mixture of saturated hydrocarbon and an alkyl alcohol. In some embodiments, the saturated hydrocarbon is cyclohexane or methyl cyclohexane. In some embodiments, the alkyl alcohol is methanol, ethanol, n-propyl alcohol, isopropyl alcohol, n-butanol, t-butanol, pentanol, or hexanol.

In some embodiments, the organic solvent is a mixture of methyl cyclohexane and isopropyl alcohol.

In some embodiments, the heating of step (ii) is heating to a temperature of about 75-85° C. In other embodiments, the heating of step (ii) is heating to about 80° C.

In some embodiments, the cooling and stirring of step (iii) is first cooling and stirring at 25-30° C., followed by further cooling and stirring at 0° C. In some embodiments, the first cooling and stirring at 25-30° C. is for about 1-5 hours. In other embodiments, the further cooling and stirring at 0° C. is for about 1-3 hours.

In some embodiments, the slurry from the process of the invention for Form C is isolated by filtration.

In one embodiment, this invention provides a process for the preparation of a crystalline Form D of Tapinarof. The starting materials in the process are produced by any suitable method, including synthesis methods known in the art.

In some embodiments, the process of the present invention produces a high purity of the crystalline Form D of Tapinarof. In some embodiments, the chemical purity of the crystalline polymorph (Form D) of Tapinarof is from about 85% to about 100% by weight. In some embodiments, the chemical purity of the crystalline polymorph (Form D) of Tapinarof is from about 90% to about 100% by weight. In some embodiments, the chemical purity of the crystalline polymorph (Form D) of Tapinarof is from about 95% to about 100% by weight. In another embodiment, the chemical purity of the crystalline polymorph (Form D) of Tapinarof is from about 96% to about 100% by weight. In another embodiment, the chemical purity of the crystalline polymorph (Form D) of Tapinarof is from about 97% to about 100% by weight. In another embodiment, the chemical purity of the crystalline polymorph (Form D) of Tapinarof is from about 98% to about 100% by weight. In another embodiment, the chemical purity of the crystalline polymorph (Form D) of Tapinarof is from about 99% to about 100% by weight. In another embodiment, the crystalline polymorph (Form D) of Tapinarof has a chemical purity of about 99% by weight. In some embodiments, the crystalline polymorph (Form B) of Tapinarof of the invention has a chemical purity of at least 99% by weight.

In one embodiment, this invention provides a crystalline polymorph (Form D) of Tapinarof, which is prepared by the process of the invention as described herein.

According to an embodiment of the present invention, there is provided a process for preparing Tapinarof crystalline Form D, said process comprising:
(i) mixing crude Tapinarof and an organic solvent to provide a mixture,
(ii) heating and stirring the mixture of step (i) to obtain a solution,
(iii) cooling and stirring the solution to form a slurry, and
(iv) isolating the slurry and drying to obtain the crystalline Form D.

In some embodiments, the organic solvent for the process of the invention for Form D is a mixture of saturated hydrocarbon and an ether. In some embodiments, the saturated hydrocarbon is cyclohexane or methyl cyclohexane. In some embodiments, the ether is diethyl ether, methyl ethyl ether, or methyl t-butyl ether (MTBE). In some embodiments, the organic solvent is a mixture of methyl cyclohexane and methyl t-butyl ether (MTBE).

In some embodiments, the heating of step (ii) is heating to a temperature of about 75-85° C. In other embodiments, the heating of step (ii) is heating to about 80° C.

In some embodiments, the cooling and stirring of step (iii) is first cooling and stirring at 25-30° C., followed by further cooling and stirring at 0° C. In some embodiments, the first cooling and stirring at 25-30° C. is for about 1-5 hours. In other embodiments, the further cooling and stirring at 0° C. is for about 1-4 hours.

In some embodiments, the slurry from the process of the invention for Form D is isolated by filtration.

In some embodiments, the crude Tapinarof is prepared by the process comprising:
(i) mixing (E)-2-isopropyl-1,3-dimethoxy-5-styrylbenzene (IDSB) with pyridine hydrochloride in N-methyl-2-pyrrolidone (NMP) to provide a mixture,
(ii) heating the mixture of step (i) to 160° C. and stirring,
(iii) adding methyl t-butyl ether (MTBE) to the mixture of step (ii) and performing a phase separation, and
(v) washing the organic phase with HCl solution and water.

In some embodiments, the present invention provides pharmaceutical compositions comprising a crystalline polymorph of the present invention, e.g., a crystalline Form B of Tapinarof, a crystalline Form C of Tapinarof, or a crystalline Form D of Tapinarof, or a Tapinarof compound of the invention, and at least one pharmaceutically acceptable excipient.

It will be understood by one of skill in the art that the percentage of a particular form of Tapinarof is expressed in relation to all forms of Tapinarof present in a sample. For example, the phrase “Tapinarof having at least 95% crystalline Form B” is meant to convey that at least 95% of all forms of the Tapinarof present is in Form B.

In one embodiment, the pharmaceutical composition comprises a crystalline Form B of Tapinarof of the invention and an additional solid-state form of Tapinarof, and at least one pharmaceutically acceptable excipient.

In another embodiment, the pharmaceutical composition comprises a crystalline Form C of Tapinarof of the invention and an additional solid-state form of Tapinarof, and at least one pharmaceutically acceptable excipient.

In another embodiment, the pharmaceutical composition comprises a crystalline Form D of Tapinarof of the invention and an additional solid-state form of Tapinarof, and at least one pharmaceutically acceptable excipient.

The Tapinarof and solid-state forms thereof according to the present invention may have advantageous properties selected from at least one of the following: chemical or polymorphic purity, flowability, solubility, dissolution rate, bio-viability, morphology or crystal habit, stability-such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility, and bulk density. Each represents a separate embodiment of this invention.

A solid-state form, such as a crystal form, may be referred to herein as being characterized by graphical data “as depicted in” or “as substantially depicted in” a Figure. Such data include, for example, powder X-ray diffractograms. As is well-known in the art, the graphical data potentially provides additional technical information to further define the respective solid-state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Tapinarof referred to herein as being characterized by graphical data “as depicted in” or “as substantially depicted in” a Figure will thus be understood to include any crystal forms of Tapinarof characterized with the graphical data having such small variations, as are well known to the skilled person, in comparison with the Figure.

As used herein, the “additional solid-state form” of Tapinarof refers to any solid-state forms of Tapinarof or any solid-state forms of Tapinarof known in the art.

As used herein, and unless stated otherwise, the term “anhydrous” or “non-solvated” in relation to crystalline forms of Tapinarof, relates to a crystalline form of Tapinarof which does not include any crystalline water (“anhydrous”) or other solvents (non-solvated) in a defined, stoichiometric amount within the crystal. Moreover, an “anhydrous” or “non-solvated” form would typically, not contain more than 0.5% (w/w) of either water or organic solvents respectively as measured for example by TGA (Thermal Gravimetric Analysis).

As used herein, the term “isolated” in reference to a crystalline polymorph of Tapinarof of the present invention corresponds to a crystalline polymorph of Tapinarof that is physically separated from the reaction mixture in which it is formed.

In some embodiment, this invention provides a pharmaceutical composition comprising a combination of a crystalline Form B of Tapinarof, a crystalline Form C of Tapinarof, a crystalline Form D of Tapinarof, an amorphous Tapinarof, or different solid form of Tapinarof known in the art, including Form A, or any combination thereof, wherein Tapinarof Form A is characterized by an X-ray powder diffraction pattern exhibiting characteristic diffraction peaks at 6.9, 10.7, 16.3, 20.4 and 22.7±0.2 degrees 2θ, wherein Form A is further characterized by one or more peaks at 12.2, 12.7, 14.6, 15.1 and 17.0±0.2 degrees 2θ.

In some embodiments, the weight ratio between the crystalline Form B and other amorphous or different solid forms is in the range of between 10:1 to 1:10. In some embodiments, the weight ratio between the crystalline Form B and other amorphous or different solid forms is 1:1, 2:1, 3:1, 4:1, 5:1, 6:1, 7:1, 8:1, 9:1, 10:1, 1:10, 1:9, 1:8, 1:7, 1:6, 1:5, 1:4, 1:3 or 1:2 or any ranges thereof.

In some embodiments, the weight ratio between the crystalline Form C and other amorphous or different solid forms is in the range of between 10:1 to 1:10. In some embodiments, the weight ratio between the crystalline Form C and other amorphous or different solid forms is 1:1, 2:1, 3:1, 4:1, 5:1, 6:1, 7:1, 8:1, 9:1, 10:1, 1:10, 1:9, 1:8, 1:7, 1:6, 1:5, 1:4, 1:3 or 1:2 or any ranges thereof.

In some embodiments, the weight ratio between the crystalline Form D and other amorphous or different solid forms is in the range of between 10:1 to 1:10. In some embodiments, the weight ratio between the crystalline Form D and other amorphous or different solid forms is 1:1, 2:1, 3:1, 4:1, 5:1, 6:1, 7:1, 8:1, 9:1, 10:1, 1:10, 1:9, 1:8, 1:7, 1:6, 1:5, 1:4, 1:3 or 1:2 or any ranges thereof.

In some embodiments, the present invention provides a dosage form comprising the pharmaceutical composition as described herein, wherein said dosage form is selected from a cream, a lotion, a gel, an ointment, an emulsion, a solution, a suspension, an elixir, a tincture, a paste, a foam, an aerosol, a spray, a patch, a transdermal patch and an applicator syringe.

The crystalline Form B of Tapinarof of the invention, the crystalline Form C of Tapinarof of the invention, or the crystalline Form D of Tapinarof of the invention, or the Tapinarof compound of the invention, and the pharmaceutical compositions comprising the same of the invention are useful for the treatment, prevention or amelioration of skin disorders.

In one aspect, the present invention provides methods for the treatment, prevention and/or amelioration of skin disorders comprising administering a therapeutically effective amount of the crystalline Form B of Tapinarof of the invention, the crystalline Form C of Tapinarof of the invention, the crystalline Form D of Tapinarof of the invention, or a Tapinarof compound of the invention, or the pharmaceutical compositions comprising the same of the invention.

In some embodiments of the methods of the invention, the skin disorders are selected from psoriasis (selected from plaque psoriasis, flexural/inverse psoriasis, scalp psoriasis, sebopsoriasis, nail psoriasis and genital psoriasis); dermatitis (also known as eczema) selected from atopic dermatitis, contact dermatitis, dyshidrotic dermatitis, nummular dermatitis and seborrheic dermatitis; acne selected from acne vulgaris, papulopustular acne and nodular acne; rosacea selected from erythematotelangiectatic rosacea, papulopustular rosacea, rhinophyma rosacea and ocular rosacea; ichthyosis selected from ichthyosis vulgaris, hereditary ichthyosis, acquired ichthyosis; scaling skin; imbalance of skin barrier; hidradenitis suppurativa; tinea selected from tinea pedis, tinea capitis, tinea barbae, tinea faciei, tinea corporis, tinea manum, tinea cruris and tinea interdigital; hereditary palmoplantar keratoderma; actinic keratosis; a keratinization skin disorder; a keratinization mucosal disorder; Gorlin syndrome; Palmoplantar Keratoderma (PPK); and Olmsted syndrome by topical or intralesional administration.

In some embodiments, the present invention provides a method for treating, preventing, or alleviating a skin disorder in a subject in need thereof, comprising administering to the subject a therapeutically effective amount of a crystalline Form B, Form C, or Form D, or a Tapinarof compound of the invention, or a pharmaceutical composition, or a dosage form of the invention. In some embodiments, the crystalline Form B, Form C, or Form D, or the Tapinarof compound of the invention, or said pharmaceutical composition, or said dosage form is administered topically or intralesionally to the affected area of the skin disorder in said subject.

In some embodiments, the skin disorder is selected from psoriasis (selected from plaque psoriasis, flexural/inverse psoriasis, scalp psoriasis, sebopsoriasis, nail psoriasis and genital psoriasis); dermatitis (also known as eczema) selected from atopic dermatitis, contact dermatitis, dyshidrotic dermatitis, nummular dermatitis and seborrheic dermatitis; acne selected from acne vulgaris, papulopustular acne and nodular acne; rosacea selected from erythematotelangiectatic rosacea, papulopustular rosacea, rhinophyma rosacea and ocular rosacea; ichthyosis selected from ichthyosis vulgaris, hereditary ichthyosis, acquired ichthyosis; scaling skin; imbalance of skin barrier; hidradenitis suppurativa; tinea selected from tinea pedis, tinea capitis, tinea barbae, tinea faciei, tinea corporis, tinea manum, tinea cruris and tinea interdigital; hereditary palmoplantar keratoderma; actinic keratosis; a keratinization skin disorder; a keratinization mucosal disorder; Gorlin syndrome; Palmoplantar Keratoderma (PPK); and Olmsted syndrome. In some embodiments, the skin disorder is psoriasis. In some embodiments, the skin disorder is dermatitis. In some embodiments, the skin disorder is acne. In some embodiments, the skin disorder is rosacea. In some embodiments, the skin disorder is ichthyosis. In some embodiments, the skin disorder is scaling skin. In some embodiments, the skin disorder is imbalance of skin barrier. In some embodiments, the skin disorder is tinea. In some embodiments, the skin disorder is hidradenitis suppurativa. In some embodiments, the skin disorder is Palmoplantar Keratoderma (PPK).

In some embodiments, the psoriasis is plaque psoriasis, scalp psoriasis, sebopsoriasis, or genital psoriasis.

In some embodiments, the acne is acne vulgaris, papulopustular acne, or nodular acne.

In some embodiments, the rosacea is erythematotelangiectatic rosacea, papulopustular rosacea, rhinophyma rosacea, or ocular rosacea.

In some embodiments the dermatitis (eczema) is atopic dermatitis, contact dermatitis, dyshidrotic dermatitis, nummular dermatitis, or seborrheic dermatitis.

In some embodiments, the tinea is tinea pedis, tinea capitis, tinea barbae, tinea faciei, tinea corporis, tinea manum, tinea cruris, or tinea interdigital.

In some embodiments, the method of the invention comprises once daily or twice daily administration.

In some embodiments, the pharmaceutical compositions of the invention comprise a therapeutically effective amount of a crystalline Form B of Tapinarof, a crystalline Form C of Tapinarof, or a crystalline Form D of Tapinarof, or a Tapinarof compound of the invention or any combination thereof, and a suitable pharmaceutically acceptable excipient for the treatment, prevention or alleviation of the symptoms manifested by a skin disorder. In some embodiments, the pharmaceutically acceptable excipient is suitable for topical or intralesional use. In some embodiments, the present invention provides a process for preparing the pharmaceutical compositions of the invention by mixing a crystalline Form B of Tapinarof, a crystalline Form C of Tapinarof, or a crystalline Form D of Tapinarof with at least one pharmaceutically acceptable excipient.

In some embodiments of the method of the invention, the pharmaceutical composition comprises a therapeutically effective amount of the crystalline Form B of Tapinarof of the invention and at least one pharmaceutically acceptable excipient. In some embodiments, the pharmaceutical composition comprises the crystalline Form B of Tapinarof of the invention in an amount of from about 0.1% w/w to about 10.0% w/w. In some embodiments, the pharmaceutical composition comprises the crystalline Form B of Tapinarof of the invention in an amount of from about 0.25% w/w to about 10.0% w/w.

In some embodiments of the method of the invention, the pharmaceutical composition comprises the crystalline Form B of Tapinarof of the invention in an amount of from 0.25% w/w to 0.5% w/w. In some embodiments, the pharmaceutical composition comprises the crystalline Form B of Tapinarof of the invention in an amount of from 0.5% w/w to 1% w/w. In some embodiments, the pharmaceutical composition comprises the crystalline Form B of Tapinarof of the invention in an amount of from 1% w/w to 1.5% w/w. In some embodiments, the pharmaceutical composition comprises the crystalline Form B of Tapinarof of the invention in an amount of from 1.5% w/w to 2% w/w. In some embodiments, the pharmaceutical composition comprises the crystalline Form B of Tapinarof of the invention in an amount of from 2% w/w to 5% w/w. In some embodiments, the pharmaceutical composition comprises the crystalline Form B of Tapinarof of the invention in an amount of from 5% w/w to 10% w/w. In other embodiments, the pharmaceutical composition comprises the crystalline Form B of Tapinarof in an amount of 0.25%, 0.5%, 1%, or 5% w/w. Each possibility represents a separate embodiment of the present invention. In another embodiment, the composition is a topical composition. In another embodiment, the composition is an intralesional injection.

In some embodiments of the method of the invention, a Tapinarof compound of this invention is administered. In other embodiments the Tapinarof compound is about 85% pure by weight and is at least 85% Form B by weight of the present invention. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 90% Form B by weight, or at least 91% Form B by weight, or at least 92% Form B by weight, or at least 93% Form B by weight, or at least 94% Form B by weight, or at least 95% Form B by weight, or at least 96% Form B by weight, or at least 97% Form B by weight, or at least 98% Form B by weight, or at least 99% Form B by weight. In some embodiments, the compound is about 96% Form B by weight, about 97% Form B by weight, about 98% Form B by weight, or about 99% Form B by weight. In some embodiments, the compound is at least 99% Form B by weight.

In some embodiments of the method of the invention, a Tapinarof compound of this invention is administered. In other embodiments the Tapinarof compound is about 98% pure by weight and at least 95% Form B by weight. In some embodiments, the compound Tapinarof is about 96% Form B, about 97% Form B, about 98% Form B, or about 99% Form B. In some embodiments, the compound Tapinarof is at least 99% Form B. In some embodiment, the compound Tapinarof has a chemical purity of at least 85%. In other embodiments, the compound Tapinarof has a chemical purity of at least 85%, 86%, 87%, 88%, 89%, 90%, 91%, 92%, 93%, 94%, 95%, 96%, 97%, 98%, or 99%. In other embodiments, the pharmaceutical composition comprises the compound Tapinarof having at least 85%, 86%, 87%, 88%, 88%, 89%, 90%, 91%, 92%, 93%, 94%, 95%, 96%, 97%, 98%, or 99% crystalline Form B.

In some embodiments of the method of the invention, a Tapinarof compound of this invention is administered. In other embodiments the Tapinarof compound has a cis isomer content of 0.1% or less and at least 85% pure by weight, wherein the compound is at least 85% Form B by weight, characterized by an x-ray powder diffraction pattern having peaks at 7.7, 11.0, and 22.6 (±0.2) ° 2θ. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 85% Form B by weight, or at least 86% Form B by weight, or at least 87% Form B by weight, or at least 88% Form B by weight, or at least 89% Form B by weight, or at least 90% Form B by weight, or at least 91% Form B by weight, or at least 92% Form B by weight, or at least 93% Form B by weight, or at least 94% Form B by weight, or at least 95% Form B by weight, or at least 96% Form B by weight, or at least 97% Form B by weight, or at least 98% Form B by weight, or at least 99% Form B by weight.

In some embodiments, the Tapinarof compound has a cis isomer content of 0.1% or less, wherein the compound is at least 98% pure by weight, wherein the compound is at least 85% Form B, characterized by an x-ray powder diffraction pattern having peaks at 7.7, 11.0, and 22.6 (±0.2) ° 2θ. In some embodiments, the Tapinarof compound has a cis isomer content of 0.1% or less and is at least 98% pure by weight, wherein the compound is at least 95% Form B. In other embodiments, the Tapinarof compound has at least 85%, 86%, 87%, 88%, 89%, 90%, 91%, 92%, 93%, 94%, 95%, 96%, 97%, 98%, or 99% crystalline Form B.

In some embodiments of the method of the invention, the pharmaceutical composition comprises a therapeutically effective amount of the crystalline Form C of Tapinarof of the invention and at least one pharmaceutically acceptable excipient. In some embodiments, the pharmaceutical composition comprises the crystalline Form C of Tapinarof of the invention in an amount of from about 0.1% w/w to about 10.0% w/w. In some embodiments, the pharmaceutical composition comprises the crystalline Form C of Tapinarof of the invention in an amount of from about 0.25% w/w to about 10.0% w/w.

In some embodiments of the method of the invention, the pharmaceutical composition comprises the crystalline Form C of Tapinarof of the invention in an amount of from 0.25% w/w to 0.5% w/w. In some embodiments, the pharmaceutical composition comprises the crystalline Form C of Tapinarof of the invention in an amount of from 0.5% w/w to 1% w/w. In some embodiments, the pharmaceutical composition comprises the crystalline Form C of Tapinarof of the invention in an amount of from 1% w/w to 1.5% w/w. In some embodiments, the pharmaceutical composition comprises the crystalline Form C of Tapinarof of the invention in an amount of from 1.5% w/w to 2% w/w. In some embodiments, the pharmaceutical composition comprises the crystalline Form C of Tapinarof of the invention in an amount of from 2% w/w to 5% w/w. In some embodiments, the pharmaceutical composition comprises the crystalline Form C of Tapinarof of the invention in an amount of from 5% w/w to 10% w/w. In other embodiments, the pharmaceutical composition comprises the crystalline Form C of Tapinarof in an amount of 0.25%, 0.5%, 1%, or 5% w/w. Each possibility represents a separate embodiment of the present invention. In another embodiment, the composition is a topical composition. In another embodiment, the composition is an intralesional injection.

In some embodiments of the method of the invention, a Tapinarof compound of this invention is administered. In other embodiments the Tapinarof compound is about 85% pure by weight and at least 85% Form C by weight of the invention. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 85% Form C by weight, or at least 86% Form C by weight, or at least 87% Form C by weight, or at least 88% Form C by weight, or at least 89% Form C by weight, or at least 90% Form C by weight, or at least 91% Form C by weight, or at least 92% Form C by weight, or at least 93% Form C by weight, or at least 94% Form C by weight, or at least 95% Form C by weight, or at least 96% Form C by weight, or at least 97% Form C by weight, or at least 98% Form C by weight, or at least 99% Form C by weight. In some embodiments, the compound is about 96% Form C by weight, about 97% Form C by weight, about 98% Form C by weight, or about 99% Form C by weight. In some embodiments, the compound is at least 99% Form C by weight.

In some embodiments of the method of the invention, a Tapinarof compound of this invention is administered. In other embodiments the Tapinarof compound is about 98% pure by weight and at least 95% Form C by weight. In some embodiments, the compound Tapinarof is about 96% Form C, about 97% Form C, about 98% Form C, or about 99% Form C. In some embodiments, the compound Tapinarof is at least 99% Form C. In some embodiment, the compound Tapinarof has a chemical purity of at least 85%. In other embodiments, the compound Tapinarof has a chemical purity of at least 85%, 86%, 87%, 88%, 89%, 90%, 91%, 92%, 93%, 94%, 95%, 96%, 97%, 98%, or 99%. In other embodiments, the pharmaceutical composition comprises the compound Tapinarof having at least 85%, 86%, 87%, 88%, 89%, 90%, 91%, 92%, 93%, 94%, 95%, 96%, 97%, 98%, or 99% crystalline Form C.

In some embodiments of the method of the invention, a Tapinarof compound of this invention is administered. In other embodiments the Tapinarof compound has a cis isomer content of 0.1% or less, wherein the compound is at least 85% pure by weight, wherein the compound is at least 85% Form C, characterized by an x-ray powder diffraction pattern having peaks at 5.2, 10.5, 12.6, 15.8, and 22.9 (±0.2) ° 2θ. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 85% Form C by weight, or at least 86% Form C by weight, or at least 87% Form C by weight, or at least 88% Form C by weight, or at least 89% Form C by weight, or at least 90% Form C by weight, or at least 91% Form C by weight, or at least 92% Form C by weight, or at least 93% Form C by weight, or at least 94% Form C by weight, or at least 95% Form C by weight, or at least 96% Form C by weight, or at least 97% Form C by weight, or at least 98% Form C by weight, or at least 99% Form C by weight.

In some embodiments of the method of the invention, the Tapinarof compound has a cis isomer content of 0.1% or less and is at least 98% pure by weight, wherein the compound is at least 85% Form C. In other embodiments, the Tapinarof compound has at least 85%, 86%, 87%, 88%, 89%, 90%, 91%, 92%, 93%, 94%, 95%, 96%, 97%, 98%, or 99% crystalline Form C. In some embodiments, the Tapinarof compound has a cis isomer content of 0.1% or less, wherein the compound is at least 98% pure by weight, wherein the compound is at least 95% Form C, characterized by an x-ray powder diffraction pattern having peaks at 5.2, 10.5, 12.6, 15.8, and 22.9 (±0.2) ° 2θ.

In some embodiments of the method of the invention, the pharmaceutical composition comprises a therapeutically effective amount of the crystalline Form D of Tapinarof of the invention and at least one pharmaceutically acceptable excipient. In some embodiments, the pharmaceutical composition comprises the crystalline Form D of Tapinarof of the invention in an amount of from about 0.1% w/w to about 10.0% w/w. In some embodiments, the pharmaceutical composition comprises the crystalline Form D of Tapinarof of the invention in an amount of from about 0.25% w/w to about 10.0% w/w.

In some embodiments of the method of the invention, the pharmaceutical composition comprises the crystalline Form D of Tapinarof of the invention in an amount of from 0.25% w/w to 0.5% w/w. In some embodiments, the pharmaceutical composition comprises the crystalline Form D of Tapinarof of the invention in an amount of from 0.5% w/w to 1% w/w. In some embodiments, the pharmaceutical composition comprises the crystalline Form D of Tapinarof of the invention in an amount of from 1% w/w to 1.5% w/w. In some embodiments, the pharmaceutical composition comprises the crystalline Form D of Tapinarof in an amount of from 1.5% w/w to 2% w/w. In some embodiments, the pharmaceutical composition comprises the crystalline Form D of Tapinarof in an amount of from 2% w/w to 5% w/w. In some embodiments, the pharmaceutical composition comprises the crystalline Form D of Tapinarof in an amount of from 5% w/w to 10% w/w. In other embodiments, the pharmaceutical composition comprises the crystalline Form D of Tapinarof in an amount of 0.25%, 0.5%, 1%, or 5% w/w. Each possibility represents a separate embodiment of the present invention. In another embodiment, the composition is a topical composition. In another embodiment, the composition is an intralesional injection.

In some embodiments of the method of the invention, a Tapinarof compound of this invention is administered. In other embodiments the Tapinarof compound is at least 85% pure by weight and at least 85% Form D by weight of the invention. In some embodiments, the compound is at least 86% pure by weight, is at least 87% pure by weight, is at least 88% pure by weight, is at least 89% pure by weight, is at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 85% Form D by weight, or at least 86% Form D by weight, or at least 87% Form D by weight, or at least 88% Form D by weight, or at least 89% Form D by weight, or at least 90% Form D by weight, or at least 91% Form D by weight, or at least 92% Form D by weight, or at least 93% Form D by weight, or at least 94% Form D by weight, or at least 95% Form D by weight, or at least 96% Form D by weight, or at least 97% Form D by weight, or at least 98% Form D by weight, or at least 99% Form D by weight. In some embodiments, the compound is about 96% Form D by weight, about 97% Form D by weight, about 98% Form D by weight, or about 99% Form D by weight. In some embodiments, the compound is at least 99% Form D by weight.

In some embodiments of the method of the invention, a Tapinarof compound of this invention is administered. In other embodiments the Tapinarof compound is about 98% pure by weight and at least 95% Form D by weight. In some embodiments, the compound Tapinarof is about 96% Form D, about 97% Form D, about 98% Form D, or about 99% Form D. In some embodiments, the compound Tapinarof is at least 99% Form D. In some embodiment, the compound Tapinarof has a chemical purity of at least 90%. In other embodiments, the compound Tapinarof has a chemical purity of at least 85%, 86%, 87%, 88%, 89%, 90%, 91%, 92%, 93%, 94%, 95%, 96%, 97%, 98%, or 99%. In other embodiments, the pharmaceutical composition comprises the compound Tapinarof having at least 85%, 86%, 87%, 88%, 89%, 90%, 91%, 92%, 93%, 94%, 95%, 96%, 97%, 98%, or 99% crystalline Form D.

In some embodiments of the method of the invention, a Tapinarof compound of this invention is administered. In other embodiments the Tapinarof compound has a cis isomer content of 0.1% or less, wherein the compound is at least 85% pure by weight, wherein the compound is at least 85% Form D, characterized by an x-ray powder diffraction pattern having peaks at 8.8, 10.8, 13.2, 19.8, and 20.1 (±0.2) ° 2θ. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In some embodiments, the compound is at least 90% Form D by weight, or at least 91% Form D by weight, or at least 92% Form D by weight, or at least 93% Form D by weight, or at least 94% Form D by weight, or at least 95% Form D by weight, or at least 96% Form D by weight, or at least 97% Form D by weight, or at least 98% Form D by weight, or at least 99% Form D by weight.

In some embodiments of the method of the invention, the Tapinarof compound has a cis isomer content of 0.1% or less and is at least 98% pure by weight, wherein the compound is at least 85% Form D. In other embodiments, the Tapinarof compound has at least 85%, 86%, 87%, 88%, 89%, 90%, 91%, 92%, 93%, 94%, 95%, 96%, 97%, 98%, or 99% crystalline Form D. In some embodiments, the Tapinarof compound has a cis isomer content of 0.1% or less, wherein the compound is at least 98% pure by weight, wherein the compound is at least 95% Form D, characterized by an x-ray powder diffraction pattern having peaks at 8.8, 10.8, 13.2, 19.8, and 20.1 (±0.2) ° 2θ.

### Forms II-IV

In some embodiments, the present invention provides a Tapinarof compound, wherein the compound has a cis isomer content of 0.1% or less, wherein the compound is at least 85% pure by weight, wherein the compound is at least 85% Form II by weight, characterized by an x-ray powder diffraction pattern having peaks at 7.2, 14.5, 18.0, 19.8, 22.0 and 23.5 (±0.2) ° 2θ. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In some embodiments, the compound is at least 99% pure by weight. In other embodiments, the compound is at least 85% Form II by weight, or at least 86% Form II by weight, or at least 87% Form II by weight, or at least 88% Form II by weight, or at least 89% Form II by weight, or at least 90% Form II by weight, or at least 91% Form II by weight, or at least 92% Form II by weight, or at least 93% Form II by weight, or at least 94% Form II by weight, or at least 95% Form II by weight, or at least 96% Form II by weight, or at least 97% Form II by weight, or at least 98% Form II by weight, or at least 99% Form II by weight.

In some embodiments, the present invention provides a Tapinarof compound, wherein the compound has a cis isomer content of 0.1% or less, wherein the compound is at least 85% pure by weight, wherein the compound is at least 85% Form III, characterized by an x-ray powder diffraction pattern having peaks at 5.8, 11.5, 12.5, 14.1, 16.0 and 17.3 (±0.2) ° 2θ. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In other embodiments, the compound is at least 99% pure by weight. In some embodiments, the compound is at least 85% Form III by weight, or at least 86% Form III by weight, or at least 87% Form III by weight, or at least 88% Form III by weight, or at least 89% Form III by weight, or at least 90% Form III by weight, or at least 91% Form III by weight, or at least 92% Form III by weight, or at least 93% Form III by weight, or at least 94% Form III by weight, or at least 95% Form III by weight, or at least 96% Form III by weight, or at least 97% Form III by weight, or at least 98% Form III by weight, or at least 99% Form III by weight.

In some embodiments, the present invention provides a Tapinarof compound, wherein the compound has a cis isomer content of 0.1% or less, wherein the compound is at least 85% pure by weight, wherein the compound is at least 85% Form IV, characterized by an x-ray powder diffraction pattern having peaks at 12.1, 13.3, 16.0, 20.0, 24.3 and 27.1 (±0.2) ° 2θ. In some embodiments, the compound is at least 85% pure by weight, or at least 86% pure by weight, or at least 87% pure by weight, or at least 88% pure by weight, or at least 89% pure by weight, or at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight. In other embodiments, the compound is at least 99% pure by weight. In some embodiments, the compound is at least 85% Form IV by weight, or at least 86% Form IV by weight, or at least 87% Form IV by weight, or at least 88% Form IV by weight, or at least 89% Form IV by weight, or at least 90% Form IV by weight, or at least 91% Form IV by weight, or at least 92% Form IV by weight, or at least 93% Form IV by weight, or at least 94% Form IV by weight, or at least 95% Form IV by weight, or at least 96% Form IV by weight, or at least 97% Form IV by weight, or at least 98% Form IV by weight, or at least 99% Form IV by weight.

Pharmaceutical acceptable excipients suitable for preparation of the compositions provided herein include any such excipients, carriers or vehicles known to those skilled in the art to be suitable for the particular mode of administration.

Pharmaceutical acceptable excipients, carriers or vehicles suitable for preparation of the compositions provided herein include any such excipients known to those skilled in the art to be suitable for topical administration or intralesional administration.

The resulting compositions may be a solution, suspension, emulsion or the like and are formulated as creams, gels, ointments, emulsions, solutions, elixirs, lotions, suspensions, tinctures, pastes, foams, aerosols, sprays, patches, foams or any other formulation suitable for topical administration.

Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include those suited for use include lotions, creams, solutions, gels, tapes and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

The resulting composition may be a lotion, a solution, a suspension, an emulsion or the like and is formulated as creams, gels, ointments, emulsions, solutions, elixirs, lotions, suspensions, tinctures, pastes, foams, aerosols, sprays, patches, foams, sebum control products or any other formulation suitable for topical administration. The preferred compositions are the cream, the lotion, the gel and the foam.

Pharmaceutical carriers or vehicles suitable for administration of the compounds provided herein include any such carriers known to those skilled in the art to be suitable for the particular mode of administration.

Sebum control products may include ingredients selected from azelaic acid, salicylic acid, sulfur, nicotinamide, L-carnitine and combinations thereof.

Suitable pharmaceutically and dermatologically acceptable excipients, vehicles for topical application include lotions, creams, foams, solutions, gels, patches and the like. Generally, the vehicle is either organic in nature or an aqueous emulsion and capable of accommodating the selected active agent(s), which may be micronized, dispersed, suspended or dissolved therein. The vehicle may include pharmaceutically acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

According to an aspect of the invention, there is provided a method of treatment, prevention or alleviation of a skin or mucosal disorder selected from psoriasis (selected from plaque psoriasis, flexural/inverse psoriasis, scalp psoriasis, sebopsoriasis, nail psoriasis and genital psoriasis); dermatitis (also known as eczema) selected from atopic dermatitis, contact dermatitis, dyshidrotic dermatitis, nummular dermatitis and seborrheic dermatitis; acne selected from acne vulgaris, papulopustular acne and nodular acne; rosacea selected from erythematotelangiectatic rosacea, papulopustular rosacea, rhinophyma rosacea and ocular rosacea; ichthyosis selected from ichthyosis vulgaris, hereditary ichthyosis, acquired ichthyosis; scaling skin; imbalance of skin barrier; hidradenitis suppurativa; tinea selected from tinea pedis, tinea capitis, tinea barbae, tinea faciei, tinea corporis, tinea manum, tinea cruris and tinea interdigital; hereditary palmoplantar keratoderma; actinic keratosis; a keratinization skin disorder; a keratinization mucosal disorder; Gorlin syndrome; Palmoplantar Keratoderma (PPK); and Olmsted syndrome, by topical administration to a subject in need thereof a therapeutically effective amount of the composition and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and a pre-filled applicator syringe. In some embodiments, the dosage form comprises the pharmaceutical composition of this invention wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

The present invention further provides methods of using a crystalline Form B of Tapinarof, a crystalline Form C of Tapinarof, or a crystalline Form D of Tapinarof of the present invention, or a Tapinarof compound of the invention, in the treatment of diseases or conditions including diseases or conditions for which Tapinarof provides therapeutic benefit to a mammal having the disease or condition, such as topical treatment of skin disorder, by administering to a subject in need thereof a therapeutically effective amount of any one of a crystalline Form B of Tapinarof, a crystalline Form C of Tapinarof, and a crystalline Form D of Tapinarof or a Tapinarof compound of the invention. In another embodiment, said skin disorder is selected from psoriasis (selected from plaque psoriasis, flexural/inverse psoriasis, scalp psoriasis, sebopsoriasis, nail psoriasis and genital psoriasis); dermatitis (also known as eczema) selected from atopic dermatitis, contact dermatitis, dyshidrotic dermatitis, nummular dermatitis and seborrheic dermatitis; acne selected from acne vulgaris, papulopustular acne and nodular acne; rosacea selected from erythematotelangiectatic rosacea, papulopustular rosacea, rhinophyma rosacea and ocular rosacea; ichthyosis selected from ichthyosis vulgaris, hereditary ichthyosis, acquired ichthyosis; scaling skin; imbalance of skin barrier; hidradenitis suppurativa; tinea selected from tinea pedis, tinea capitis, tinea barbae, tinea faciei, tinea corporis, tinea manum, tinea cruris and tinea interdigital; hereditary palmoplantar keratoderma; actinic keratosis; a keratinization skin disorder; a keratinization mucosal disorder; Gorlin syndrome; Palmoplantar Keratoderma (PPK); and Olmsted syndrome.

The present invention provides a method of treatment, prevention or alleviation of a skin disorder selected from plaque, guttate, flexural/inverse, pustular or erythrodermic psoriasis and atopic dermatitis, by once daily or twice daily topical or intralesional administration to a subject in need thereof of the pharmaceutical composition of this invention, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

In some embodiments, the present invention provides a topical composition comprising from about 0.25% w/w to about 10.0% w/w crystalline Form B of Tapinarof and at least one pharmaceutically acceptable excipient for topical or intralesional administration in the treatment, prevention or alleviation of a skin disorder selected from psoriasis (selected from plaque psoriasis, flexural/inverse psoriasis, scalp psoriasis, sebopsoriasis, nail psoriasis and genital psoriasis); dermatitis (also known as eczema) selected from atopic dermatitis, contact dermatitis, dyshidrotic dermatitis, nummular dermatitis and seborrheic dermatitis; acne selected from acne vulgaris, papulopustular acne and nodular acne; rosacea selected from erythematotelangiectatic rosacea, papulopustular rosacea, rhinophyma rosacea and ocular rosacea; ichthyosis selected from ichthyosis vulgaris, hereditary ichthyosis, acquired ichthyosis; scaling skin; imbalance of skin barrier; hidradenitis suppurativa; tinea selected from tinea pedis, tinea capitis, tinea barbae, tinea faciei, tinea corporis, tinea manum, tinea cruris and tinea interdigital; hereditary palmoplantar keratoderma; actinic keratosis; a keratinization skin disorder; a keratinization mucosal disorder; Gorlin syndrome; Palmoplantar Keratoderma (PPK); and Olmsted syndrome. In some embodiment, provided herein, a method of treatment, prevention or alleviation of a skin disorder, by topical administration to the affected area of a skin disorder subject in need thereof of a therapeutically effective amount of a composition, comprising from about 0.25% w/w to about 10.0% w/w crystalline Form B of Tapinarof.

In some embodiments, the present invention provides a topical composition comprising from about 0.25% w/w to about 10.0% w/w crystalline Form C of Tapinarof and at least one pharmaceutically acceptable excipient for topical or intralesional administration in the treatment, prevention or alleviation of a skin disorder selected from psoriasis (selected from plaque psoriasis, flexural/inverse psoriasis, scalp psoriasis, sebopsoriasis, nail psoriasis and genital psoriasis); dermatitis (also known as eczema) selected from atopic dermatitis, contact dermatitis, dyshidrotic dermatitis, nummular dermatitis and seborrheic dermatitis; acne selected from acne vulgaris, papulopustular acne and nodular acne; rosacea selected from erythematotelangiectatic rosacea, papulopustular rosacea, rhinophyma rosacea and ocular rosacea; ichthyosis selected from ichthyosis vulgaris, hereditary ichthyosis, acquired ichthyosis; scaling skin; imbalance of skin barrier; hidradenitis suppurativa; tinea selected from tinea pedis, tinea capitis, tinea barbae, tinea faciei, tinea corporis, tinea manum, tinea cruris and tinea interdigital; hereditary palmoplantar keratoderma; actinic keratosis; a keratinization skin disorder; a keratinization mucosal disorder; Gorlin syndrome; Palmoplantar Keratoderma (PPK); and Olmsted syndrome. In some embodiment, provided herein, a method of treatment, prevention or alleviation of a skin disorder, by topical administration to the affected area of a skin disorder subject in need thereof of a therapeutically effective amount of a composition, comprising from about 0.25% w/w to about 10.0% w/w crystalline Form C of Tapinarof.

In some embodiments, the present invention provides a topical composition comprising from about 0.25% w/w to about 10.0% w/w crystalline Form D of Tapinarof and at least one pharmaceutically acceptable excipient for topical or intralesional administration in the treatment, prevention or alleviation of a skin disorder selected from psoriasis (selected from plaque psoriasis, flexural/inverse psoriasis, scalp psoriasis, sebopsoriasis, nail psoriasis and genital psoriasis); dermatitis (also known as eczema) selected from atopic dermatitis, contact dermatitis, dyshidrotic dermatitis, nummular dermatitis and seborrheic dermatitis; acne selected from acne vulgaris, papulopustular acne and nodular acne; rosacea selected from erythematotelangiectatic rosacea, papulopustular rosacea, rhinophyma rosacea and ocular rosacea; ichthyosis selected from ichthyosis vulgaris, hereditary ichthyosis, acquired ichthyosis; scaling skin; imbalance of skin barrier; hidradenitis suppurativa; tinea selected from tinea pedis, tinea capitis, tinea barbae, tinea faciei, tinea corporis, tinea manum, tinea cruris and tinea interdigital; hereditary palmoplantar keratoderma; actinic keratosis; a keratinization skin disorder; a keratinization mucosal disorder; Gorlin syndrome; Palmoplantar Keratoderma (PPK); and Olmsted syndrome. In some embodiment, provided herein, a method of treatment, prevention or alleviation of a skin disorder, by topical administration to the affected area of a skin disorder subject in need thereof of a therapeutically effective amount of a composition, comprising from about 0.25% w/w to about 10.0% w/w crystalline Form D of Tapinarof.

In another embodiment, the skin disorder in the method of the invention is selected from psoriasis (selected from plaque psoriasis, flexural/inverse psoriasis, scalp psoriasis, sebopsoriasis, nail psoriasis and genital psoriasis); dermatitis (also known as eczema) selected from atopic dermatitis, contact dermatitis, dyshidrotic dermatitis, nummular dermatitis and seborrheic dermatitis; acne selected from acne vulgaris, papulopustular acne and nodular acne; rosacea selected from erythematotelangiectatic rosacea, papulopustular rosacea, rhinophyma rosacea and ocular rosacea; ichthyosis selected from ichthyosis vulgaris, hereditary ichthyosis, acquired ichthyosis; scaling skin; imbalance of skin barrier; hidradenitis suppurativa; tinea selected from tinea pedis, tinea capitis, tinea barbae, tinea faciei, tinea corporis, tinea manum, tinea cruris and tinea interdigital; hereditary palmoplantar keratoderma; actinic keratosis; a keratinization skin disorder; a keratinization mucosal disorder; Gorlin syndrome; Palmoplantar Keratoderma (PPK); and Olmsted syndrome. Each represents a separate embodiment of this invention. In another embodiment, said skin disorder is selected from plaque, guttate, flexural/inverse, pustular or erythrodermic psoriasis and combinations thereof. Each represents a separate embodiment of this invention.

In another aspect, the present invention provides a method of treating a psoriasis and atopic dermatitis comprising administering to a subject in need thereof a composition comprising a crystalline polymorph (Form B) of Tapinarof of the invention. Therapeutical effective concentrations of a crystalline polymorph (Form B) of Tapinarof, for treatment, prevention or amelioration of the symptoms manifested by the skin disorder are determined by empirical methods known in the art. Exemplary dosages, strengths and concentrations of a crystalline polymorph (Form B) of Tapinarof composition administered topically, can be in the range of between 0.25-10% w/w or between 0.25-5%, 0.25-3%, 0.25-2% w/w or at 0.1%, 0.25%, 0.5%, 1%, 2%, 3%, 4%, 5%, 6%, 7%, 8%, 9%, 10% w/w.

In another aspect, the present invention provides a method of treating a psoriasis and atopic dermatitis comprising administering to a subject in need thereof a composition comprising a crystalline polymorph (Form C) of Tapinarof of the invention. Therapeutical effective concentrations of a crystalline polymorph (Form C) of Tapinarof, for treatment, prevention or amelioration of the symptoms manifested by the skin disorder are determined by empirical methods known in the art. Exemplary dosages, strengths and concentrations of a crystalline polymorph (Form C) of Tapinarof composition administered topically, can be in the range of between 0.25-10% w/w or between 0.25-5%, 0.25-3%, 0.25-2% w/w or at 0.1%, 0.25%, 0.5%, 1%, 2%, 3%, 4%, 5%, 6%, 7%, 8%, 9%, 10% w/w.

In another aspect, the present invention provides a method of treating a psoriasis and atopic dermatitis comprising administering to a subject in need thereof a composition comprising a crystalline polymorph (Form D) of Tapinarof of the invention. Therapeutical effective concentrations of a crystalline polymorph (Form D) of Tapinarof, for treatment, prevention or amelioration of the symptoms manifested by the skin disorder are determined by empirical methods known in the art. Exemplary dosages, strengths and concentrations of a crystalline polymorph (Form D) of Tapinarof composition administered topically, can be in the range of between 0.25-10% w/w or between 0.25-5%, 0.25-3%, 0.25-2% w/w or at 0.1%, 0.25%, 0.5%, 1%, 2%, 3%, 4%, 5%, 6%, 7%, 8%, 9%, 10% w/w.

The frequency of administration can be determined empirically. Exemplary frequencies are once daily, twice daily, weekly, bi-weekly or monthly. Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

In some embodiments, the present invention provides a method for the treatment of a skin disorder, wherein the treatment comprises once daily or twice daily topical or intralesional administration to a subject in need thereof of a therapeutically effective amount of a crystalline Form B of Tapinarof, a crystalline Form C of Tapinarof, or a crystalline Form D of Tapinarof or any combination thereof with a suitable pharmaceutically acceptable excipient suitable for topical use, for the treatment, prevention or alleviation of the symptoms manifested by a skin disorder.

In some embodiments, the present invention provides a regimen of administration comprising the once daily or twice daily administration to a skin disorder subject in need thereof of a therapeutically effective dose of the composition of this invention until the skin disorder is cured, prevented or alleviated.

In some embodiments, the present invention provides a regimen of administration comprising the once daily or twice daily administration to a skin disorder subject in need thereof a therapeutically effective amount of the dosage form of this invention.

In some embodiments, the present invention provides a kit comprising one or more dosage forms as described herein and instructions for use.

Pharmaceutical excipients, carriers or vehicles suitable for preparation of the compositions provided herein include any such carriers known to those skilled in the art to be suitable for the particular mode of administration.

The resulting composition may be a lotion, a solution, a suspension, an emulsion or the like and is formulated as creams, gels, ointments, emulsions, solutions, elixirs, lotions, suspensions, tinctures, pastes, foams, aerosols, sprays, patches, foams, or any other formulation suitable for topical administration. The preferred compositions are the cream, the foam and the lotion.

The resulting composition may be administered intralesional. In another embodiment the composition is an intralesional injection or microneedles.

The compositions according to the invention are pharmaceutical compositions, and especially dermatological compositions, which may be in any form conventionally used for topical application and especially in the form of lotions, creams. By addition of a fatty or oily phase, it may also be in the form of dispersions of the lotion or serum type, emulsions of liquid or semi-liquid consistency of the milk type obtained by dispersing a fatty phase in an aqueous phase (0/W) or conversely (W/O), or suspensions or emulsions of soft, semi-liquid or solid consistency of the cream, gel or ointment type, or alternatively multiple emulsions (W/O/W or 0/W/0), microemulsions, microcapsules, microparticles or vesicular dispersions of ionic and/or nonionic type, or wax/aqueous phase dispersions.

The composition according to the invention is an intralesional composition. In another embodiment, intralesional administration is done by regular injections or with microneedles. The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents. Such sterile formulations may be prepared using a non-toxic parenterally acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Pharmaceutical compositions that are useful in the methods of the invention may be administered, prepared, packaged, and/or sold in formulations suitable for intralesional delivery. The compositions may contain pharmaceutically acceptable excipients/carriers and other ingredients known to enhance and facilitate drug administration. Other possible formulations, such as nanoparticles, liposomes may also be used.

In other embodiments, said composition further comprises at least one fatty alcohol. In yet other embodiments, said composition further comprises a polyacrylic acid homopolymer or copolymer.

In some embodiments, said water in said oil in water emulsion further comprises at least one water soluble humectant.

In other embodiments, said at least one water soluble humectant is selected from the group consisting of propylene glycol, glycerin, and polyethylene glycol-X, where X is in the range of 200 to 10,000. Each possibility represents a separate embodiment of the present invention.

In some embodiments, the crystalline Form of Tapinarof of the invention, e.g., a crystalline Form B, a crystalline Form C, or a crystalline Form D, encapsulated in a microcapsule, where “microcapsule” refers to a microparticle having a core shell structure, wherein said core comprises a crystalline Form of Tapinarof as defined herein, being coated by a shell forming the microcapsule entrapping the core.

In further embodiments, the compositions of this invention for treating skin disorder are controlled or slowed release drug delivery system, wherein the active agent is encapsulated, coated, adsorbed, embedded, impregnated, dispersed, entrapped, or encased in a polymeric material and providing a sustained release formulation.

When referring to a “controlled or slowed release drug delivery system” it should be understood to relate to a delivery system (which in the present invention is a topical delivery system) that enables the release of the Tapinarof in predetermined amounts over a specified period. In some embodiments said system is a core-shell system of a microcapsule or a porous matrix structure, such as for example a microsponge.

The term “embedded” should be understood to encompass an inert system that provides a barrier between the Tapinarof, and its surrounding environment in the composition. In some embodiments said agent is entrapped and/or encapsulated in said controlled release system.

The term “about” in reference to a numerical value stated herein is to be understood as the stated value+/−10%.

### Analytical Methods

### Powder X-Ray Diffraction XRD Method

As used herein, unless stated otherwise, the XRPD measurements are taken using Bruker D8 Advance powder X-ray diffractometer equipped with LYNXEYE XE-T detector, utilizing CuKα radiation, with step size of 0.02° 2θ and count time of 0.30 seconds per step.

### Differential Scanning Calorimetry (DSC) Method

DSC data was collected using Mettler-Toledo make and model is TGA/DSC1 STAR System. DSC performed on 3.7442 mg of Solid sample in crucible. Heating range: 40-400° C., heating rate: 10°/minute.

### Thermal Gravimetric Analysis (TGA) Method

TGA data was collected using Mettler-Toledo make and model is TGA/DSC1 STAR System. TGA performed on 4.1753 mg of solid sample in crucible. Heating range: 30-400° C., heating rate: 20°/minute.

### EXAMPLES

The following examples further illustrate the invention but should not be construed as in any way limiting its scope.

### Example 1: Preparation of Crystalline Form B of Tapinarof

The present invention discloses novel crystalline form B of Tapinarof (also known as Benvitimod).

Form B is characterized by the following characteristic XRPD peaks: 7.7, 11.0, 22.6±0.2 ° 2θ.

Form B can further be characterized by the above peaks and one or more of the following peaks: 6.1, 9.9, 15.5, 20.0, 21.9±0.2° 2θ.

Form B could not be obtained polymorphically pure. FIG. 1 presents the XRPD pattern of the polymorphic mixture containing form B, the characteristic peaks of form B are marked by “B” in FIG. 1.

For B is anhydrous, non-solvated form.

40 g of TPNF were added to a reactor with 400 mL of Methyl Cyclohexane and 32 mL of Isopropyl alcohol. The solution was heated to 80° C. and stirred for 1 hour. The reactor was cooled to 25° C. for 4 hours and stirred at 25° C. for 1 hour. The reactor was cooled to 0° C. for 1 hour and stirred at 0° C. for 2 hours. The slurry was filtered, and the cake was washed with 40 mL of Methyl Cyclohexane twice. The product was dried at 50° C. under vacuum conditions for 10 hours.

### Example 2: Preparation of Crystalline Form B of Tapinarof

40 g of TPNF were added to a reactor with 400 mL of Methyl Cyclohexane and 32 mL of Isopropyl alcohol. The solution was heated to 80° C. and stirred for 1 hour. The reactor was cooled to 25° C. for 4 hours and stirred at 25° C. for 1 hour. The reactor was cooled to 0° C. for 1 hour and stirred at 0° C. for 2 hours. The slurry was filtered, and the cake was washed with 80 mL of Methyl Cyclohexane twice. The product was dried at 50° C. under vacuum conditions for 10 hours.

### Example 3: Preparation of Crystalline Form C of Tapinarof

The current invention discloses novel crystal form C of Tapinarof (also known as Benvitimod). Form C is characterized by the following characteristic XRPD peaks: 5.2, 10.5, 12.6, 15.8, 22.9±0.2° 2θ.

Form C can further be characterized by the above peaks and one or more of the following peaks: 11.4, 25.5, 26.5, 27.1, 31.6±0.2° 2θ.

Form C could not be obtained polymorphically pure. FIG. 2 presents the XRPD pattern of the polymorphic mixture containing Form C, the characteristic peaks of form C are marked by “C” in FIG. 2.

Form C is an isopropanol solvate.

25 g of TPNF were added to a reactor with 250 mL of Methyl Cyclohexane and 20 mL of Isopropyl alcohol. The solution was heated to 80° C. and stirred for 1 hour. The reactor was cooled to 25° C. for 4 hours and stirred at 25° C. for 1 hour. The reactor was cooled to 0° C. for 1 hour and stirred at 0° C. for 2 hours. The slurry was filtered.

X-ray diffraction was measured using Panalytical X-ray diffractometer model X'Pert Pro using CuKα radiation.

Measurement Parameters:

Start Position (° 2θ): 3; End Position (° 2θ): 40; Step Size: 0.004° 2θ;

Scan Step Time: 10 sec.

### Example 4: Preparation of Crystalline Form D of Tapinarof

The current invention discloses novel crystalline form D of Tapinarof (also known as Benvitimod).

Form D is characterized by the following characteristic XRPD peaks: 8.8, 10.8, 13.2, 19.8, 20.1±0.2° 2θ. From D can further be characterized by the above peaks and one or more of the following peaks: 4.4, 16.5, 18.6, 21.7, 26.5±0.2° 2θ.

Form D could not be obtained polymorphically pure. FIG. 3 presents the XRPD pattern of the polymorphic mixture containing form C, the characteristic peaks of form D are marked by “D” in FIG. 3.

Form D is a methyl t-butyl ether solvate.

415 g of TPNF were added to a reactor with 4150 mL of Methyl Cyclohexane and 581 mL of methyl t-butyl ether (MTBE). The solution was heated to 80° C. and stirred for 1 hour. The reactor was cooled to 25° C. for 4 hours and stirred at 25° C. for 1 hour. The reactor was cooled to 0° C. for 1.5 hours and stirred at 0° C. for 2 hours. The slurry was filtered, and the cake was washed with 415 mL of Methyl Cyclohexane twice.

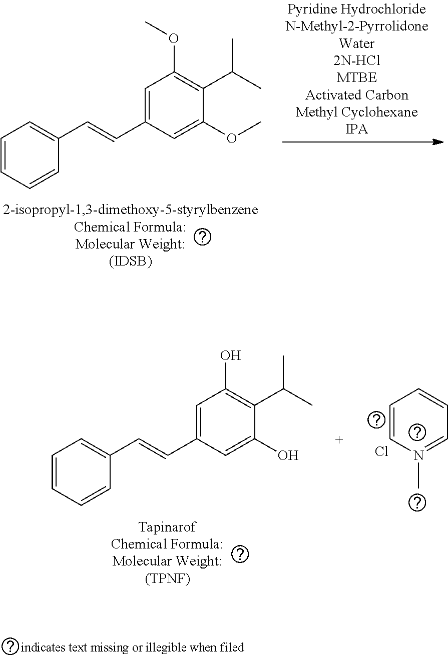

### Example 5: Preparation of Crystalline Form D of Tapinarof

1.30 kg of IDSB ((E)-2-isopropyl-1,3-dimethoxy-5-styrylbenzene) were added to a reactor with 4.25 kg of Pyridine Hydrochloride and 1.30 L of N-methyl-2-pyrrolidone (NMP). The reactor was heated to 160° C. and stirred for 14 hours. 7.8 L of water and 3.9 L of methyl t-butyl ether (MTBE) were added to the reactor and a phase separation was performed. The aqueous phase was extracted with MTBE and the two organic phases were mixed. The organic phase was washed twice with HCl solution and three times with water. The remained organic phase was treated with active Carbone. 1.73 L of Methyl Cyclohexane were added to the solution and 6.5 L of distillate was distilled out.

6.5 L of Methyl Cyclohexane and 1.04 L of MTBE were added to the reactor and the reactor was heated to 80° C. The reactor was stirred at 80° C. for two hours. The reactor was cooled to 25° C. for 4 hours and stirred at 25° C. for 1 hour. The reactor was cooled to 0° C. for 1 hour and stirred at 0° C. for 2 hours. The slurry was filtered, and the cake was washed with 1.3 L of Methyl Cyclohexane twice.

X-ray diffraction was measured using Panalytical X-ray diffractometer model X'Pert Pro using CuKα radiation.

Measurement Parameters:

Start Position (° 2θ): 3; End Position (° 2θ): 40; Step Size: 0.004° 2θ;

Scan Step Time: 10 sec.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A crystalline Form B of Tapinarof, characterized by an X-ray powder diffraction pattern exhibiting characteristic diffraction (XRPD) peaks at 7.7, 11.0, and 22.6 (±0.2) ° 2θ.

2. The crystalline Form B of Tapinarof according to claim 1, further characterized by one or more peaks at 6.1, 9.9, 15.5, 20.0, and 21.9 (±0.2) ° 2θ.

3. The crystalline Form B of Tapinarof according to claim 1, having the X-ray powder diffraction peak positions and intensities as depicted in FIG. 1.

4. The crystalline Form B of Tapinarof according to claim 1, wherein said crystalline Form B is an anhydrous form.

5. The crystalline Form B of Tapinarof according to claim 1, wherein said crystalline Form B is a non-solvated form.

6. A pharmaceutical composition comprising a pharmaceutically effective amount of the crystalline Form B of Tapinarof according to claim 1 and at least one pharmaceutically acceptable excipient.

7. The pharmaceutical composition according to claim 6, wherein said composition comprises the crystalline Form B of Tapinarof in an amount of from about 0.1% w/w to about 10.0% w/w.

8. The pharmaceutical composition according to claim 7, wherein said composition comprises the crystalline Form B of Tapinarof in an amount of 0.25%, 0.5%, 1%, or 5% w/w.

9. A dosage form comprising the pharmaceutical composition according to claim 6, wherein said dosage form is selected from a cream, a lotion, a gel, an ointment, an emulsion, a solution, a suspension, an elixir, a tincture, a paste, a foam, an aerosol, a spray, a patch, a transdermal patch and an applicator syringe.

10. A kit comprising one or more dosage forms according to claim 9 and instructions for use.

11. A crystalline Form C of Tapinarof, characterized by an X-ray powder diffraction pattern exhibiting characteristic diffraction (XRPD) peaks at 5.2, 10.5, 12.6, 15.8, and 22.9 (±0.2) ° 2θ.

12. The crystalline Form C of Tapinarof according to claim 11, further characterized by one or more peaks at 11.4, 25.5, 26.5, 27.1, 31.6 (±0.2) ° 2θ.

13. The crystalline Form C of Tapinarof according to claim 11, having the X-ray powder diffraction peak positions and intensities as depicted in FIG. 2.

14. The crystalline Form C of Tapinarof according to claim 11, wherein said crystalline Form C is an isopropanol solvate form.

15. The crystalline Form C of Tapinarof according to claim 11, wherein said crystalline Form C is an anhydrous form.

16. A pharmaceutical composition comprising a pharmaceutically effective amount of the crystalline Form C of Tapinarof according to claim 11 and at least one pharmaceutically acceptable excipient.

17. The pharmaceutical composition according to claim 16, wherein said composition comprises the crystalline Form C of Tapinarof in an amount of from about 0.25% w/w to about 10.0% w/w.

18. The pharmaceutical composition according to claim 17, wherein said composition comprises the crystalline Form C of Tapinarof in an amount of 0.25%, 0.5%, 1%, or 5% w/w.

19. A dosage form comprising the pharmaceutical composition according to claim 16, wherein said dosage form is selected from a cream, a lotion, a gel, an ointment, an emulsion, a solution, a suspension, an elixir, a tincture, a paste, a foam, an aerosol, a spray, a patch, a transdermal patch and an applicator syringe.

20. A kit comprising one or more dosage forms according to claim 19 and instructions for use.

21. A crystalline Form D of Tapinarof, characterized by an X-ray powder diffraction pattern exhibiting characteristic diffraction (XRPD) peaks at 8.8, 10.8, 13.2, 19.8, and 20.1 (±0.2) ° 2θ.

22. The crystalline Form D of Tapinarof according to claim 21, further characterized by one or more peaks at 4.4, 16.5, 18.6, 21.7, and 26.5 (±0.2) ° 2θ.

23. The crystalline Form D of Tapinarof according to claim 21, having the X-ray powder diffraction peak positions and intensities as depicted in FIG. 3.

24. The crystalline Form D of Tapinarof according to claim 21, wherein said crystalline Form D is a methyl t-butyl ether solvate form.

25. The crystalline Form D of Tapinarof according to claim 21, wherein said crystalline Form D is an anhydrous form.

26. A pharmaceutical composition comprising a pharmaceutically effective amount of the crystalline Form D of Tapinarof according to claim 21 and at least one pharmaceutically acceptable excipient.

27. The pharmaceutical composition according to claim 26, wherein said composition comprises the crystalline Form D of Tapinarof in an amount of from about 0.25% w/w to about 10.0% w/w.

28. The pharmaceutical composition according to claim 27, wherein said composition comprises the crystalline Form D of Tapinarof in an amount of 0.25%, 0.5%, 1%, or 5% w/w.

29. A dosage form comprising the pharmaceutical composition according to claim 26, wherein said dosage form is selected from a cream, a lotion, a gel, an ointment, an emulsion, a solution, a suspension, an elixir, a tincture, a paste, a foam, an aerosol, a spray, a patch, a transdermal patch and an applicator syringe.

30. A kit comprising one or more dosage forms according to claim 29 and instructions for use.

31. A Tapinarof compound, wherein the compound is about 85% pure by weight and wherein the compound is at least 85% Form B by weight according to claim 1.

32. The compound according to claim 31, wherein the compound is at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight.

33. The compound according to claim 31, wherein the compound is at least 99% pure by weight.

34. The compound according to claim 31, wherein the compound is at least 90% Form B by weight, or at least 91% Form B by weight, or at least 92% Form B by weight, or at least 93% Form B by weight, or at least 94% Form B by weight, or at least 95% Form B by weight, or at least 96% Form B by weight, or at least 97% Form B by weight, or at least 98% Form B by weight, or at least 99% Form B by weight.

35. The compound according to claim 31, wherein the compound is about 96% Form B by weight, about 97% Form B by weight, about 98% Form B by weight, or about 99% Form B by weight.

36. The compound according to claim 31, wherein the compound is at least 99% Form B by weight.

37. A Tapinarof compound, wherein the compound has a cis isomer content of 0.1% or less, wherein the compound is at least 85% pure by weight, wherein the compound is at least 85% Form B by weight, characterized by an x-ray powder diffraction pattern having peaks at 7.7, 11.0, and 22.6 (±0.2) ° 2θ.

38. The compound according to claim 37, wherein the compound is at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight.

39. The compound according to claim 38, wherein the compound is at least 99% pure by weight.

40. The compound according to claim 37, wherein the compound is at least 90% Form B by weight, or at least 91% Form B by weight, or at least 92% Form B by weight, or at least 93% Form B by weight, or at least 94% Form B by weight, or at least 95% Form B by weight, or at least 96% Form B by weight, or at least 97% Form B by weight, or at least 98% Form B by weight, or at least 99% Form B by weight.

41. A Tapinarof compound, wherein the compound is about 85% pure by weight and wherein the compound is at least 85% Form C by weight according to claim 11.

42. The compound according to claim 41, wherein the compound is at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight.

43. The compound according to claim 42, wherein the compound is at least 99% pure by weight.

44. The compound according to claim 41, wherein the compound is at least 90% Form C by weight, or at least 91% Form C by weight, or at least 92% Form C by weight, or at least 93% Form C by weight, or at least 94% Form C by weight, or at least 95% Form C by weight, or at least 96% Form C by weight, or at least 97% Form C by weight, or at least 98% Form C by weight, or at least 99% Form C by weight.

45. The compound according to claim 41, wherein the compound is about 96% Form C by weight, about 97% Form C by weight, about 98% Form C by weight, or about 99% Form C by weight.

46. The compound according to claim 41, wherein the compound is at least 99% Form C by weight.

47. A Tapinarof compound, wherein the compound has a cis isomer content of 0.1% or less, wherein the compound is at least 85% pure by weight, wherein the compound is at least 85% Form C, characterized by an x-ray powder diffraction pattern having peaks at 5.2, 10.5, 12.6, 15.8, and 22.9 (±0.2) ° 2θ.

48. The compound according to claim 47, wherein the compound is at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight.

49. The compound according to claim 47, wherein the compound is at least 99% pure by weight.

50. The compound according to claim 47, wherein the compound is at least 90% Form C by weight, or at least 91% Form C by weight, or at least 92% Form C by weight, or at least 93% Form C by weight, or at least 94% Form C by weight, or at least 95% Form C by weight, or at least 96% Form C by weight, or at least 97% Form C by weight, or at least 98% Form C by weight, or at least 99% Form C by weight.

51. A Tapinarof compound, wherein the compound is at least 85% pure by weight and wherein the compound is at least 85% Form D by weight according to claim 21 and a pharmaceutically acceptable excipient.

52. The compound according to claim 51, wherein the compound is at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight.

53. The compound according to claim 52, wherein the compound is at least 99% pure by weight.

54. The compound according to claim 51, wherein the compound is at least 90% Form D by weight, or at least 91% Form D by weight, or at least 92% Form D by weight, or at least 93% Form D by weight, or at least 94% Form D by weight, or at least 95% Form D by weight, or at least 96% Form D by weight, or at least 97% Form D by weight, or at least 98% Form D by weight, or at least 99% Form D by weight.

55. The compound according to claim 51, wherein the compound is about 96% Form D by weight, about 97% Form D by weight, about 98% Form D by weight, or about 99% Form D by weight.

56. The compound according to claim 51, wherein the compound is at least 99% Form D by weight.

57. A Tapinarof compound, wherein the compound has a cis isomer content of 0.1% or less, wherein the compound is at least 85% pure by weight, wherein the compound is at least 85% Form D, characterized by an x-ray powder diffraction pattern having peaks at 8.8, 10.8, 13.2, 19.8, and 20.1 (±0.2) ° 2θ.

58. The compound according to claim 57, wherein the compound is at least 90% pure by weight, or at least 91% pure by weight, or at least 92% pure by weight, or at least 93% pure by weight, or at least 94% pure by weight, or at least 95% pure by weight, or at least 96% pure by weight, or at least 97% pure by weight, or at least 98% pure by weight, or at least 99% pure by weight.

59. The compound according to claim 57, wherein the compound is at least 99% pure by weight.

60. The compound according to claim 57, wherein the compound is at least 90% Form D by weight, or at least 91% Form D by weight, or at least 92% Form D by weight, or at least 93% Form D by weight, or at least 94% Form D by weight, or at least 95% Form D by weight, or at least 96% Form D by weight, or at least 97% Form D by weight, or at least 98% Form D by weight, or at least 99% Form D by weight.

61. A method for treating, preventing, or alleviating a skin disorder in a subject in need thereof, comprising administering to the subject a pharmaceutically effective amount of a crystalline Form B according to claim 1.

62. A method for treating, preventing, or alleviating a skin disorder in a subject in need thereof, comprising administering to the subject a pharmaceutically effective amount of a crystalline Form C according to claim 11.

63. A method for treating, preventing, or alleviating a skin disorder in a subject in need thereof, comprising administering to the subject a pharmaceutically effective amount of a crystalline Form C according to claim 21.
